(19) United States
(12) Patent Application Publication
(10) Pub. No.: US 2011/0144641 A1
(43) Pub. Date: Jun. 16, 2011

(54) High-Intensity Pulsed Electric Field Vitrectomy Apparatus

(75) Inventors: Ramon Carsola Dimalanta, JR., Trabuco Canyon, CA (US); Tammo Heeren, Aliso Viejo, CA (US); John Christopher Huculak, Mission Viejo, CA (US); Steven W. Kovalcheck, Aliso Viejo, CA (US); Fred Mercado, Laguna Hills, CA (US)

(73) Assignee: ALCON RESEARCH, LTD., Fort Worth, TX (US)

(21) Appl. No.: 12/967,146

(22) Filed: Dec. 14, 2010

Related U.S. Application Data
(60) Provisional application No. 61/286,453, filed on Dec. 15, 2009.

(51) Int. Cl. A61F 9/007

(52) U.S. Cl. 606/45

(57) ABSTRACT

A high-intensity pulsed electric field (HIPEF) vitrectomy apparatus is disclosed. An exemplary apparatus includes a HIPEF probe comprising at least one electrode disposed at a distal end of the HIPEF probe, such that the distal end is configured for insertion into an eye. Various embodiments include a probe shaft assembled from a modular end segment and one or more additional modular shaft segments, each of the modular end segment and one or more modular shaft segments in turn comprising at least two longitudinal channels adapted to accommodate an electrode unit. In some of these probe shafts, each of the modular end segment and one or more modular shaft segments has a central longitudinal channel, and the probe shaft further comprises an aspiration tube disposed within the central longitudinal channel.

### PRIORITY CLAIM

This application claims the benefit of priority of U.S. Provisional Patent Application Ser. No. 61/286,453, filed on Dec. 15, 2009.

### TECHNICAL FIELD

The present invention relates generally to the field of eye surgery and more particularly to methods and apparatus for performing eye surgery using high-intensity pulsed electric fields.

### BACKGROUND

Techniques and apparatus for dissociation and removal of highly hydrated macroscopic volumes of proteinaceous tissue from the human eye have been previously disclosed. In particular, techniques for dissociation and removal of highly hydrated macroscopic volumes of proteinaceous tissue using rapid variable direction energy field flow fractionation have been disclosed by Steven W. Kovalcheck in “System For Dissociation and Removal of Proteinaceous Tissue”, U.S. patent application Ser. No. 11/608,877, filed 11 Dec. 2006 and published 5 Jul. 2007 as U.S. Patent Application Publ. No. 2007/0156129 (hereinafter “the Kovalcheck application”), the entire contents of which are incorporated herein by reference.

The techniques disclosed in the Kovalcheck application were described in detail in terms of vitreoretinal surgery. However, those of ordinary skill in the art will readily understand that those techniques are applicable to medical procedures in other areas in the body of humans or animals. As explained in the Kovalcheck application, prior art procedures for vitreoretinal posterior surgery have relied for decades on mechanical or traction methods such as: 1) tissue removal with shear cutting probes (utilizing either a reciprocating or rotary cutter); 2) membrane transection using scissors, a blade, or vitreous cutters; 3) membrane peeling with forceps and picks; and 4) membrane separation with forceps and viscous fluids. While improvements in mechanisms, materials, quality, manufacturability, system support, and efficacy have progressed, many of the significant advancements in posterior intraocular surgical outcomes have been primarily attributable to the knowledge, fortitude, skill, and dexterity of the operating ophthalmic physicians.

However, the Kovalcheck application disclosed novel apparatus and methods for delivering a variable direction, pulsed high-intensity and ultrashort duration disruptive electric field (low energy) at a pulse duration, repetition rate, pulse pattern, and pulse train length tuned to the properties of the components of the intraocular extracellular matrix (ECM) to create tissue dissociation. In particular, the Kovalcheck application described a probe for delivering the pulsed rapid disruptive energy field to soft proteinaceous tissue surrounded by the probe. Once the adhesive mechanism between tissue constituents are compromised, fluidic techniques may be used to remove the dissociated tissue.

### SUMMARY

As described more fully below, embodiments of the present invention include a high-intensity pulsed electric field (HIPEF) vitrectomy apparatus that includes a HIPEF probe comprising at least one electrode disposed at a distal end of the HIPEF probe, such that the distal end is configured for insertion into an eye. In particular, various probe shaft configurations are described, including a probe shaft assembled from a modular end segment and one or more additional modular shaft segments, each of the modular end segment and one or more modular shaft segments in turn comprising at least two longitudinal channels adapted to accommodate an electrode unit. In some of these probe shafts, each of the modular end segment and one or more modular shaft segments has a central longitudinal channel, and the probe shaft further comprises an aspiration tube disposed within the central longitudinal channel. One or more of the modular end segment and the modular shaft segments may be formed from a ceramic or ceramic hybrid material, in some embodiments. In some embodiments, at least one of the modular shaft segments may include a shunting irrigation channel that provides fluid communication between an irrigation lumen and an aspiration lumen, to improve aspiration flow. More generally, at least one of the modular end segment or the modular shaft segments comprises a by-pass orifice, in some embodiments, wherein the by-pass orifice is adapted to permit fluid flow between first and second longitudinal channels in the segment.

In some embodiments of the probe shafts disclosed herein, the modular end segment and the one or more modular shaft segments each comprise at least one longitudinal channel configured to serve as an irrigation channel. In these and other embodiments, the modular end segment and the one or more modular shaft segments may each comprise at least one longitudinal channel configured to accommodate an optical waveguide. In some embodiments, the probe shaft further comprises a sheath fitted over the modular end segment and the one or more modular shaft segments.

Several probe shafts having grounding features incorporated therein are also disclosed, including embodiments in which the sheath comprises a conductive element substantially encircling the probe shaft at or near the distal end of the probe shaft, wherein the conductive element is adapted for connection to electrical ground through the body of the probe. In some of these embodiments, this conductive element comprises braided wire.

In several of the disclosed embodiments, electrode units disposed in one or more longitudinal channels of the probe shaft are configured to slide along the longitudinal channels, under user control, from a first configuration, in which a tip of each electrode unit is within or proximal to a distal end of the probe shaft, to a second configuration, in which the tip of each electrode unit is extended from the distal end of the probe. In several of the embodiments, the electrode units are pre-bent and arranged within the corresponding longitudinal channels such that the tips of the electrode units move apart in a radial direction as the electrode units are moved from the first configuration to the second configuration.

In various embodiments of a probe according to the invention, the probe comprises an integral amplifier circuit configured to amplify an electrical pulse supplied to the probe prior to application to the eye via at least one of the electrode units. In these and other embodiments, the probe may further comprise an integral termination circuit configured to receive electrical pulses supplied to the probe via a transmission line, such that the termination circuit is substantially matched to the characteristic impedance of the transmission line. In still other embodiments, the probe may further comprise a pulse-shaping circuit configured to shorten the rise time of one or more electrical pulses supplied to the probe prior to application of the shaped pulses to the eye.

Configurations involving the use of two or more probes are also disclosed. For example, an apparatus for applying high-intensity pulsed electric fields to intraocular tissue may comprise first and second probes configured to be applied to an eye, the first probe comprising at least one electrode adapted for delivery of high-intensity pulsed electric field energy to intraocular tissue. The second probe in this apparatus comprises at least one intraocular surgical feature selected from the group consisting of: an optical waveguide extending at least substantially to the distal end of the second probe; an aspiration lumen; an irrigation lumen; and one or more additional electrodes. At least one of the probes comprises an optical waveguide extending at least substantially to the distal end of the at least one of the probes.

In some embodiments of this apparatus, the first probe comprises a probe shaft assembled from a modular end segment and one or more additional modular shaft segments, each of the modular end segment and one or more modular shaft segments comprising at least two longitudinal channels adapted to accommodate an electrode unit. Some embodiments further comprise a third probe configured to be applied to the eye, the third probe comprising at least one intraocular surgical feature selected from the group consisting of: an optical waveguide extending at least substantially to the distal end of the second probe; an aspiration lumen; and an irrigation lumen.

In some of these apparatus, the first probe comprises a probe shaft having a distal end configured for insertion into the eye and further comprises a conductive element substantially encircling the distal end of the probe shaft, wherein the conductive element is adapted for connection to electrical ground through a body of the first probe. In some of these and other embodiments, the first probe may comprise two or more electrodes configured to slide along longitudinal channels in a shaft of the first probe, under user control, from a first configuration, in which a tip of each electrode unit is within or proximal to a distal end of the probe shaft, to a second configuration, in which the tip of each electrode unit is extended from the distal end of the probe. The electrode units in these embodiments may be pre-bent and arranged within the corresponding longitudinal channels such that the tips of the electrode units move apart in a radial direction as the electrode units are moved from the first configuration to the second configuration. Further, the body of the first probe may comprise various pulse processing circuits, such as one or more of an amplifier circuit configured to amplify an electrical pulse supplied to the probe for application to the eye via the at least one electrode, an integral termination circuit configured to receive electrical pulses supplied to the probe via a transmission line, wherein the termination circuit is substantially matched to the characteristic impedance of the transmission line, and/or a pulse-shaping circuit configured to shorten the rise time of one or more electrical pulses supplied to the probe prior to application of the shaped pulses to the eye.

Of course, those skilled in the art will appreciate that the present invention is not limited to the above features, advantages, contexts or examples, and will recognize additional features and advantages upon reading the following detailed description and upon viewing the accompanying drawings.

### BRIEF DESCRIPTION OF THE DRAWINGS

FIG. 1 is a perspective view of an exemplary probe used for intraocular posterior surgery.

FIG. 2 is an enlarged perspective view of the tip of the probe of FIG. 1.

FIG. 3 is a schematic diagram of a high-intensity pulsed electric field (HIPEF) vitrectomy apparatus according to some embodiments of the invention.

FIG. 4 illustrates modular components of a probe shaft according to some embodiments of the present invention.

FIG. 5 is a perspective view of a partially assembled probe shaft.

FIG. 6 illustrates details of the partially assembled probe shaft of FIG. 5.

FIG. 7 illustrates another partially assembled probe shaft and a probe shaft sheath.

FIG. 8 illustrates details of a probe shaft having extensible electrodes.

FIG. 9 illustrates side views of the probe shaft of FIG. 8.

FIG. 10 illustrates details of a probe shaft having a conductive element encircling the probe shaft near its distal end.

FIG. 11A illustrates details of another probe shaft having a conductive element encircling the probe shaft.

FIG. 11B illustrates another embodiment of a probe shaft having a conductive element encircling the probe shaft.

FIGS. 12A, 12B, and 12C illustrate several exemplary shapes for a HIPEF probe shaft.

FIG. 13 illustrates several exemplary tip configurations for a HIPEF probe.

FIGS. 14A-14E provide views of several exemplary electrode configurations, according to some embodiments of the invention.

FIGS. 15A and 15B illustrate exemplary shaft configurations that include an aspiration orifice disposed on a sidewall of the shaft.

FIGS. 16A and 16B illustrate additional shaft configurations with varying electrode tip shapes.

FIG. 17 illustrates electric field intensities corresponding to several exemplary electrode configurations.

FIG. 18 provides graphs illustrating electrical field intensities corresponding to several exemplary electrode configurations.

FIG. 19 is a chart illustrating a figure-of-merit for various electrode configurations.

### DETAILED DESCRIPTION

The present disclosure describes an apparatus and method for the dissociation and removal of highly hydrated macroscopic volumes of proteinaceous tissues, such as vitreous and intraocular tissue, during vitreoretinal surgery. More particularly, the techniques disclosed below are directed to methods and apparatus for detecting whether a high-intensity pulsed electric field (HIPEF) probe used for such surgery is actually positioned in an eye, before enabling the application of pulsed energy to the surgical site. Although the techniques disclosed herein are described in detail in terms of instruments and methods for traction-free removal of vitreous and intraocular membranes from the posterior region of the eye without damaging the ultra-fine structure and function of the adjacent or adherent retina, those of ordinary skill in the art will understand the applicability of the disclosed invention for other medical procedures on both humans and animals.

As mentioned above, the Kovalcheck application (U.S. patent application Ser. No. 11/608,877) described a new approach to performing vitreoretinal surgery, using a high-intensity directionally changing ultra-short electrical field rather than classical mechanical means historically used to engage, decompose, and remove vitreous and intraocular tissues. The Kovalcheck application was based on the discovery that a transient change in tissue condition caused by the application of a high-intensity directionally changing ultra-short electrical field is satisfactory for removal of macroscopic volumes of proteinaceous tissue. The technical success of mechanical and liquefying means supports the contention that vitreous material need not be obliterated or disrupted on a molecular level to be removed—rather, an innocuous macroscopic change of state is all that is needed for tissue removal. Accordingly, the removal of intraocular tissue enabled by the techniques described in the Kovalcheck application is traction-free.

The apparatus and method disclosed in the Kovalcheck application cause a local temporary dissociation of the adhesive and structural relations in components of intraocular proteinaceous tissue, through the application of a rapidly changing electrical field. This localized dissociation of the adhesive and structural relations between components of intraocular proteinaceous tissue enables tractionless detachment between intraocular tissue components and the retinal membrane. Fluidic techniques (irrigation and aspiration) may be utilized during the tissue dissociation process to enhance the formation of a high-intensity ultra-short-pulsed electrical field and to remove disrupted tissue at the moment of dissociation. In general, it is intended that only the material within the applied high-intensity ultra-short-pulsed electrical field (also denoted high-intensity pulsed electric field, or HIPEF, herein) is assaulted and removed. Therefore, because only the material assaulted by the applied ultra-short pulses receives the high-intensity ultra-short-pulsed electrical field, there is no far-field effect during the tissue extraction process. This high-intensity ultrashort-pulsed electrical field assault leads to dissociation of the entrained macroscopic volume of intraocular proteinaceous tissue, and then aspiration removes the dissociated entrained macroscopic volume of tissue.

Generally speaking, then, a probe (or probes) with two or more electrodes is inserted into the target hydrated tissue, vitreous or intraocular tissue. The ends of the electrodes are exposed at the distal end of the probe. An electrical pulse is transmitted down at least one of the electrodes while the other one or more electrodes act as the return conductors. A non-plasma electrical field is created between the delivery electrode(s) and the return electrode(s). With each electric pulse, the direction of the created electrical field is changed by reversing polarity of the electric pulse, by electrode switching, or by a combination of both. Pulses may be grouped into bursts, which may be repeated at different frequencies and/or different amplitudes. Such pulse groups may be directed at heterogeneous tissue. The electrical pulse amplitude, duration, duty cycle and repetition rate along with continual changing of field direction, create a disruptive electrical field across the orifice of the aspiration lumen. Tissue is drawn into the orifice of the aspiration lumen by fluidic techniques (aspiration). The tissue is then mixed or diluted with irrigation fluid and disassociated as it traverses the high-intensity ultra-short-pulsed directionally changing electric field. During a given interval, disorder is created in the electrical field by changing the direction of the electrical field between one or more of the electrodes at the tip of the probe.

The affected medium between the electrode terminations at the end of the probe consists of a mix of target tissue (e.g. vitreous) and supplemental fluid (irrigation fluid). The electrical impedance of this target medium in which the electrical field is created is maintained by the controlled delivery of supplemental fluid (irrigation fluid). In some embodiments, the supplemental fluid providing the electrical impedance is a conductive saline. The supplemental fluid may be provided by an irrigation source external to the probe, through one or more lumens within the probe or a combination of both. When the supplemental fluid is provided within and constrained to the probe interior, the supplemental fluid may have properties (e.g. pH) and ingredients (e.g. surfactants or enzymes) that may be conducive to protein dissociation.

The properties of the generated electrical energy field within the target medium are important. In the techniques disclosed in the Kovalcheck application and expanded upon herein, high-intensity, ultrashort pulses (sub-microseconds) of electrical energy are used. Tissue impedance, conductivity and dilution are maintained in the target medium by supplemental fluid irrigation, in some embodiments. The pulse shape, the pulse repetition rate, and the pulse train length may be tuned to the properties of the intraocular tissues, in some embodiments. In some embodiments, multiple pulse patterns may be employed to address the heterogeneity of intraocular tissue.

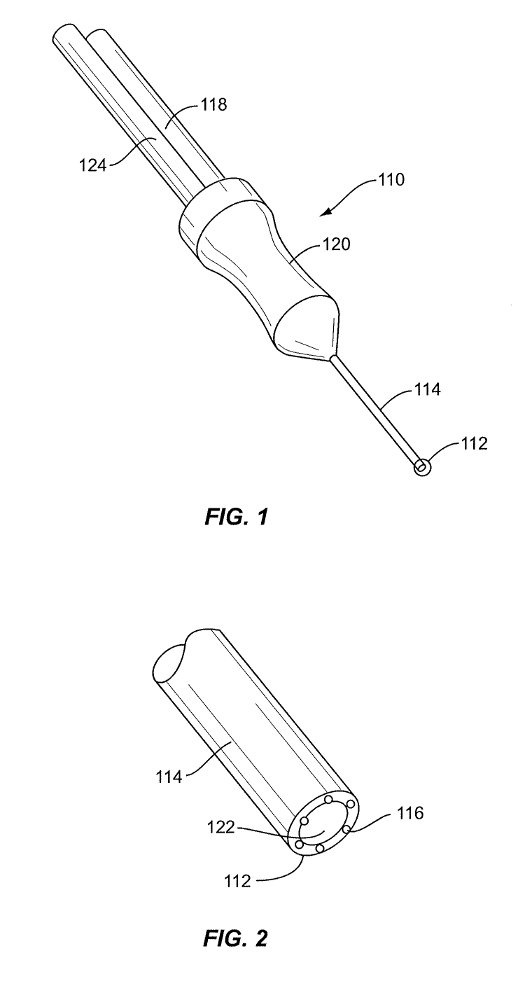

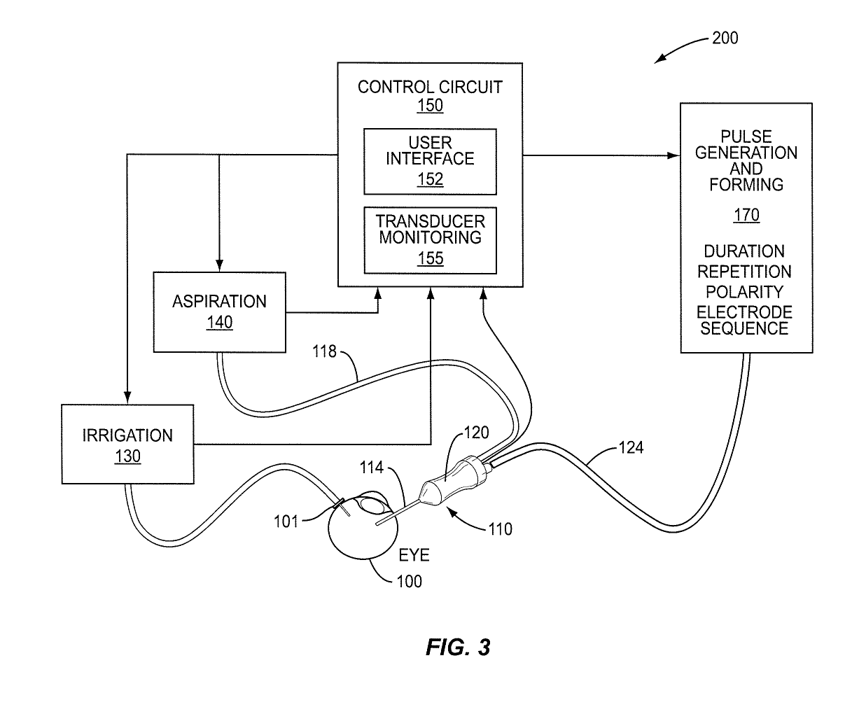

One application of the system described herein is for the treatment of pathologic retinal conditions. An exemplary apparatus for this treatment is shown in FIG. 1, which illustrates a HIPEF probe 110 comprising a hollow probe shaft 114 extending from handle 120 to probe shaft tip 112, an aspiration line 118, and electrical cable/transmission line 124. FIG. 2 illustrates details of the probe shaft 114 and probe shaft tip 112; a plurality of electrodes 116, connected to electrical cable 124, are exposed at the tip 112, and surround an aspiration lumen 122 providing an aspiration pathway to aspiration tube 118.

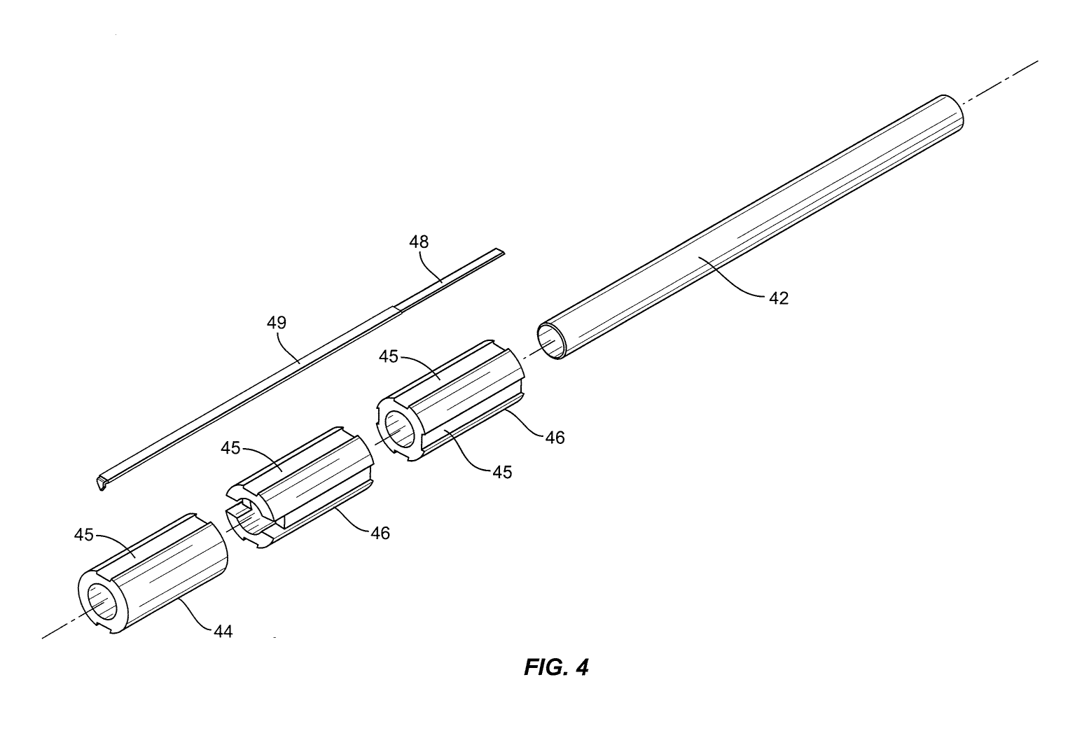

The tip 112 of probe 110 may be inserted by a surgeon into the posterior region of an eye 100 via a pars plana approach 101, as shown in FIG. 3, using handle 120. Using a standard visualization process, vitreous and/or intraocular membranes and tissues are engaged by the tip 112 at the distal end of the hollow probe 114, irrigation 130 and aspiration 140 mechanisms are activated, and ultra-short high-intensity pulsed electric energy is delivered from a pulse generation and forming circuit 170 through cable 124 (which may comprise a transmission line, for example), creating a disruptive high-intensity ultra-short-pulsed electrical field within the entrained volume of tissue. The adhesive mechanisms of the entrained constituents of the tissue that are drawn toward the probe tip 112 via aspiration through an aspiration line 118 connected to an aspiration lumen 122 in the hollow probe 114 are dissociated, and disrupted tissue removed with the aid of the employed fluidic techniques. Engagement may be axial to or lateral to the tip 112 of the hollow probe 114; extracted tissue is removed through the aspiration lumen 122 via a saline aspiration carrier to a collection module.

The apparatus pictured in FIGS. 1 to 3 delivers high-intensity pulsed electric fields (HIPEF) at a pulse duration, repetition rate, pulse pattern, and pulse train length tuned to the properties of the components of the intraocular extracellular matrix. The pulse generation and forming circuit 170 for the system 200 pictured in FIG. 3 delivers pulsed DC or gated AC against a low impedance presented by the vitreous and the irrigating solution. Included in the pulse generation circuit 170 are energy storage, pulse shaping, transmission, and load-matching components. In some embodiments, the peak output voltage of the pulse generation circuit 170 is sufficient to deliver up to a 300 kV/cm field strength using the electrodes 116 at the distal end 112 of the hollow surgical probe 114 (see FIG. 2). The pulse duration is short relative to the dielectric relaxation time of protein complexes. Further, the pulse duration, repetition rate, and pulse train length (i.e., duty cycle) are chosen to avoid the development of thermal effects (“cold” process). Thus, in some embodiments the system 200 generates and delivers square-shaped or trapezoidal-shaped pulses with rise and fall times of less than five nanoseconds. In some embodiments of the apparatus and method disclosed herein, pulse durations are in the nanosecond range, with voltages produced by pulse generation circuit 170 greater than one kilovolt and in some cases in the tens of kilovolts.

Control circuit 150 is configured to control the operation of pulse generation and forming circuit 170, setting pulse parameters such as pulse duration and repetition rate, and in some embodiments is configured to generate a stepwise continual change in the direction of the electrical field by directing the pulse generation and forming circuit 170 to switch between electrodes, reversing polarity between electrodes or a combination of both in an array of electrodes at the tip 112 of the hollow probe 114. This continual change in the direction of the electrical field creates disorder in the electric field without causing dielectric breakdown of the carrier fluid between the electrodes or thermal effects. In addition to a processor and appropriate software stored in device memory (not shown), control circuit 150 includes a user interface 152, which may comprise one or more of a keypad, keyboard, touchscreen, display, and the like, configured to allow an operator to select an operating mode and/or to set or adjust various pulse parameters. In addition, control circuit 150 may include, in some embodiments, a transducer monitoring circuit 155, which is configured to monitor feedback signals from the probe 110, the irrigation system 130, and/or the aspiration system 140, and to adjust and/or enable or disable application of pulse energy in response to the feedback signals.

In some embodiments, pulse shaping functions, which may include amplification, attenuation, termination, sharpening, softening, or matching of the pulses produced by the pulse generation circuit 170, are performed entirely within the pulse generation circuit 170, i.e., outside of the probe itself. In other embodiments, however, one or more of these pulse shaping functions may be moved inside the probe 110. For instance, the pulses from the pulse generator 170 should generally be terminated with an appropriate matching circuit (e.g., matched to the characteristic impedance of cable 124) at or near the probe 110. This termination is necessary to reduce reflections of the power back to the generator, and to yield clean pulses. Termination can not be done at any other place other than close to the probe. In some embodiments, the matching circuit may be installed inside the body of the probe 110.

In order to deliver high voltage pulses, (e.g., 10 kilovolts or more), the cables that deliver the electrical pulses to the probe can be bulky and inflexible. While this is not a problem in a laboratory environment, practical devices should include cabling that does not put any significant strain on the device, since the surgeon needs to have maximum ease of control of the device. Electrical breakdown considerations prohibit the use of much smaller cabling if reliability is a concern. However, if the high voltage is generated inside the probe (i.e., by amplification), the power connections may be made significantly smaller since they only have to carry relatively low voltage pulses. Furthermore, the initial generation of these lower voltage pulses at pulse generation and forming circuit 170 may be greatly simplified, yielding greater flexibility in configuring the pulse shape (e.g., with regard to pulse width). Accordingly, in some embodiments a pulse amplification circuit is included inside the body of the probe 110.

In some embodiments of the apparatus of FIG. 3, the probe 110 may be an inherently disposable device that only has to deliver a limited number of uses. In this case, design options are available that could otherwise not be used, since lifetime is less of an issue for disposable devices. In some embodiments, for example, pulse-sharpening circuitry can be moved into the body of probe 110. The function of the pulse sharpening circuitry is primarily to reduce the rise-time and/or fall-time of the applied HIPEF pulses. With the addition of pulse-sharpening circuitry to probe 110, slower pulses may be produced by the pulse generator 170, and sharpened inside the probe. Slower pulses are typically easier and cheaper to generate, and the delivery of such pulses is less difficult since less care has to be taken to preserve pulse rise-time.

In various applications, the apparatus and techniques described herein may be applied to remove all of the posterior vitreous tissue, or specific detachments of vitreous tissue from the retina or other intraocular tissues or membranes could be realized. Engagement, disruption and removal of vitreous tissue, vitreoretinal membranes, and fibrovascular membranes from the posterior cavity of the eye and surfaces of the retina are critical processes pursued by vitreoretinal specialists, in order to surgically treat sight-threatening conditions such as diabetic retinopathy, retinal detachment, proliferative vitreoretinopathy, traction of modalities, penetrating trauma, epi-macular membranes, and other retinopathologies. Though generally intended for posterior intraocular surgery involving the vitreous and retina, it can be appreciated that the apparatus described herein are applicable to anterior ophthalmic treatments as well, including traction reduction (partial vitrectomy); micelle adhesion reduction; trabecular meshwork disruption, manipulation, reorganization, and/or stimulation; trabeculoplasty to treat chronic glaucoma; Schlemm's Canal manipulation, removal of residual lens epithelium, and removal of tissue trailers. Applicability of the disclosed apparatus and methods to other medical treatments will become obvious to one skilled in the art, after a thorough review of the present disclosure and the attached figures.

In the Kovalcheck application, which describes in detail the use of high-intensity pulsed electric fields for intraocular surgery, only the basics of a HIPEF probe were disclosed. The various applicator devices presented herein provide improved pulse delivery means for the system and method disclosed in the Kovalcheck application. In particular, several device embodiments are disclosed, having features that enhance the efficacious delivery, channeling and distribution of ultrashort duration high intensity pulsed electric fields to specific volumes of ocular tissue. Features are illustrated that allow versatile manipulation of the applicator and the subsequent electric field. In some embodiments, fluidic channels may be included in the probe shafts to draw in and remove the affected volume. The fluidics may include both saline irrigation and effluent aspiration.

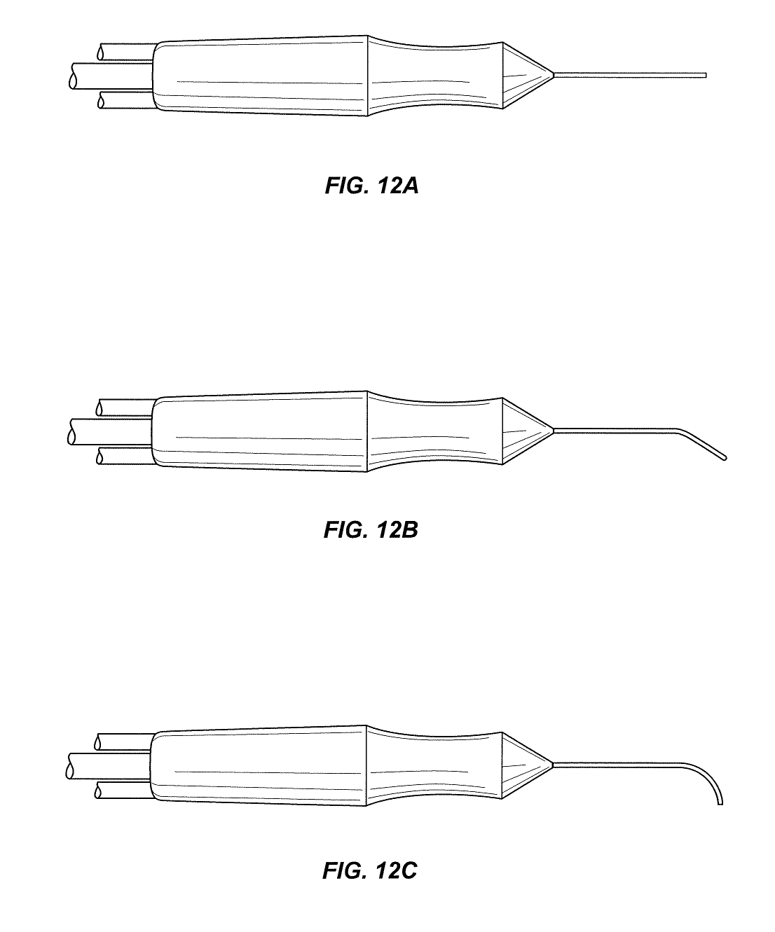

A wide variety of shaft configurations are possible. In the Kovalcheck application, for instance, a straight shaft probe was illustrated. However, since there are no moving parts in the PEF probe, many shaft shapes and configurations are possible. Several of these shapes are illustrated in FIG. 12, which illustrates a straight shaft embodiment, an angled shaft embodiment, and a curved shaft embodiment. Thus, the shaft of the applicator may be straight or contain one or more bends or curves to allow for access to difficult to reach regions of the posterior vitreous, thereby eliminating the need for scleral buckling. The radii of curves and bends along with the probe shaft reach are chosen as to better accommodate access to difficult to reach regions without placing undue stress on the scleral puncture entrance site or introducer cannula. In several embodiments, the applicator shaft may be formed from a relatively rigid material with hardness greater than 75 (shore D).

Several of the probe designs discussed herein support multiple functions, combining the electrodes used to carry HIPEF energy, one or more aspiration channels, one or more irrigation channels, etc. Thus, incorporated into the shaft of the applicator may be one or more of the following: electrical conductor(s) for passage of electric pulses with return passage of pulses provided by an independent return device or within the same probe; one or more channels for passage of optical fibers, fiber bundles, or other optical waveguide(s), for providing illumination to the distal end of the applicator, for providing optical path for a laser, and/or for imaging the ocular tissue at the distal end of the applicator; one or more “by-pass” orifices between irrigation and aspiration channels or lumens to allow shunting of low viscosity irrigation fluid to the higher viscosity aspirating effluent, thereby increasing the mobility and fluidity of the effluent (orifices may be axially spaced proximal to shaft distal face, in some embodiments, and may be graded in diameter, having diameters sequentially smaller from the distal to proximal regions of the shaft); and one or more channels for delivery of ingredient loaded irrigation fluid wherein the ingredients in the irrigation fluid contribute toward increasing the fluidity of the ocular tissue.

However, functionality may also be split between several probes. Thus, a bimanual usage scenario is possible, where independent probes or cannulas may be partnered with the pulsed field applicator. Three probes may also be used, where a third probe is passive, or fixed. Each of these independent probes or cannulas may contain one or more channels for a return electrode, optical fiber(s), aspirating effluent, delivery of irrigation or a combination thereof. The following tables provide various configurations. Tables 1A and 1B illustrate possible configurations given that a first probe (Probe 1) includes at least two electrodes for delivery of HIPEF energy; Table 1A is for a two-probe configuration while Table 1B lists options for a three-probe configuration. Each row lists probe features for probes that may be used together. For example, the third row of Table 1A illustrates that a first probe having electrodes, aspiration, and optical fiber features may be used with a second probe having irrigation capability.

TABLE 1A

| PROBE 1 | PROBE 2 |
|---|---|
| Electrodes, aspiration, irrigation | Optical fiber(s) |
| Electrodes, irrigation, optical | Aspiration |
| fiber(s) | |
| Electrodes, aspiration, optical | Irrigation |
| fiber(s) | |
| Electrodes | Aspiration, irrigation, optical fiber(s) |
| Electrodes, optical fiber(s) | Aspiration, irrigation |
| Electrodes, irrigation | Aspiration, optical fiber(s) |
| Electrodes, aspiration | Irrigation, optical fiber(s) |

TABLE 1B

| | | PROBE 3 |
|---|---|---|
| PROBE 1 | PROBE 2 | (or Cannula) |
| Electrodes | Aspiration, optical | Irrigation |
| | fiber(s) | |
| Electrodes | Aspiration, irrigation | Optical fiber(s) |
| Electrodes | Aspiration | Irrigation, optical |
| | | fiber(s) |
| Electrodes, aspiration | Optical fiber(s) | Irrigation |
| Electrodes, irrigation | Aspiration | Optical fiber(s) |
| Electrodes, optical | Aspiration | Irrigation |
| fiber(s) | | |

Configurations in which one or more electrodes are contained in each of two probes are also possible. Tables 2A and 2B illustrate the possible distribution of probe functionality for these scenarios; Table 2A is for a two-probe configuration while Table 2B lists options for a three-probe configuration. Of course, other configurations are also possible.

TABLE 2A

| PROBE 1 | PROBE 2 |
|---|---|
| Electrode(s), aspiration, irrigation | Electrode(s), optical fiber(s) |
| Electrode(s), irrigation, optical | Electrode(s), aspiration |
| fiber(s) | |
| Electrode(s), aspiration, optical | Electrode(s), irrigation |
| fiber(s) | |
| Electrode(s) | Electrode(s), aspiration, irrigation, |
| | optical fiber(s) |
| Electrode(s), optical fiber(s) | Electrode(s), aspiration, irrigation |
| Electrode(s), irrigation | Electrode(s), aspiration, optical |
| | fiber(s) |
| Electrode(s), aspiration | Electrode(s), irrigation, optical |
| | fiber(s) |

TABLE 2B

| | | PROBE 3 (or |
|---|---|---|
| PROBE 1 | PROBE 2 | Cannula) |
| Electrode(s) | Electrode(s), | Irrigation |
| | aspiration, optical fiber(s) | |
| Electrode(s) | Electrode(s), | Optical fiber(s) |
| | aspiration, irrigation | |
| Electrode(s) | Electrode(s), | Irrigation, optical |
| | aspiration | fiber(s) |
| Electrode(s), | Electrode(s), optical | Irrigation |
| aspiration | fiber(s) | |
| Electrode(s), | Electrode(s), | Optical fiber(s) |
| irrigation | aspiration | |
| Electrode(s), optical | Electrode(s), | Irrigation |
| fiber(s) | aspiration | |
| Electrode(s), | Electrode(s), | Optical fiber(s) |
| aspiration | irrigation | |

In various embodiments, the applicator body may be molded or cast, e.g., from a polymer, and includes metallic connector means to connect electrodes in the probe shaft to a cable or cables from the HIPEF pulse generator. The body may be extruded, in some embodiments, or may be a composite. The applicator shaft may be constructed from a dielectric material, a conductive material or a combination thereof. If constructed from a conductive material, the included electrode or electrodes must be insulated from the shaft. Materials and features may include ceramic or ceramic hybrids, biocompatible metals, biocompatible polymers, glass or quartz, thermoplastic (e.g. polyimide, peek), or composites of any of these. Fabrication of the shaft may be via extruding, deposition coating, molding, machining, or the shaft may be drawn, cast, formed from pressure or vacuum deposition, formed from lithography and diffusion bonding, or from two photon polymerization. Of course, combinations of these processes may be used.

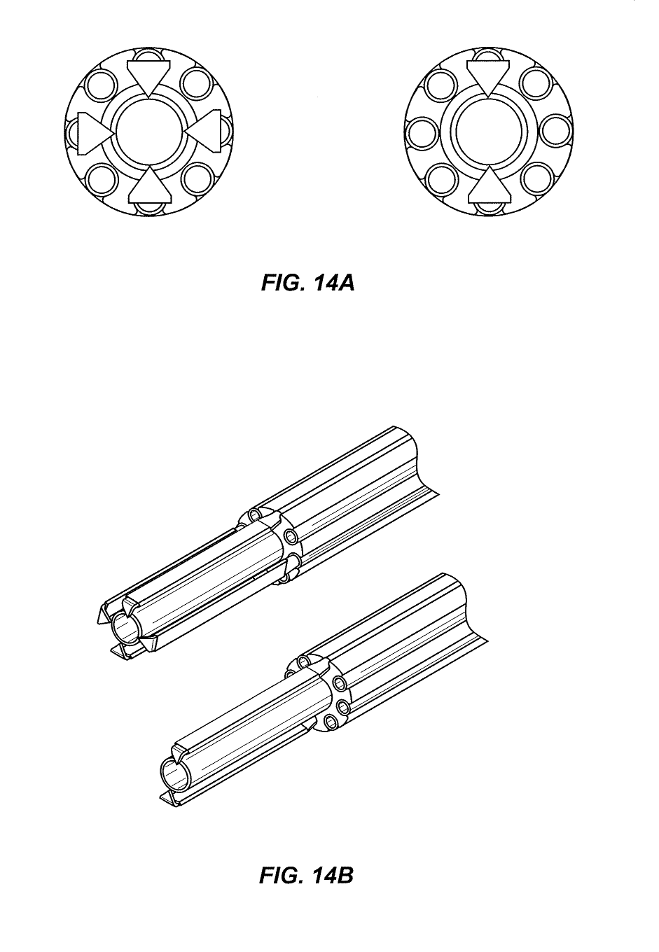

Similarly, various techniques may be used to form the electrodes used to apply the HIPEF energy to the optical tissue. For instance, a conductive material may be plated onto a dielectric shaft, deposition coated or penned onto a dielectric shaft. A wire conductor (flat or round) or penned tracing may be used. In any case, the termination may be shaped and formed to exacerbate, focus or concentrate the pulsed electric field effect induced by the HIPEF energy. A number of exemplary electrodes configurations and electrode shapes are illustrated in FIGS. 14A-14E, 15A-15B, and 16A-16B. Those skilled in the art will appreciate that additional shapes and configurations are possible.

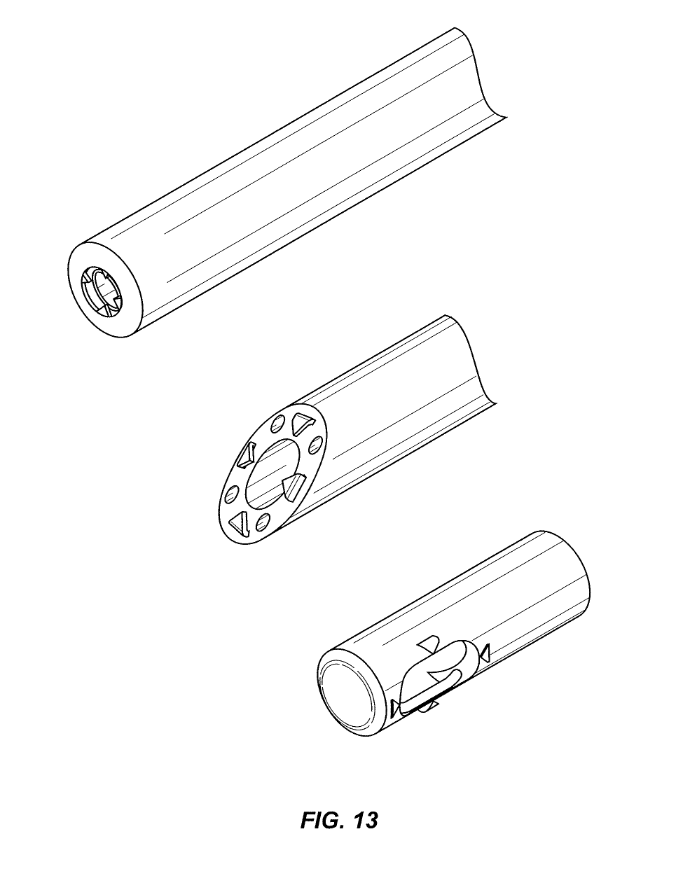

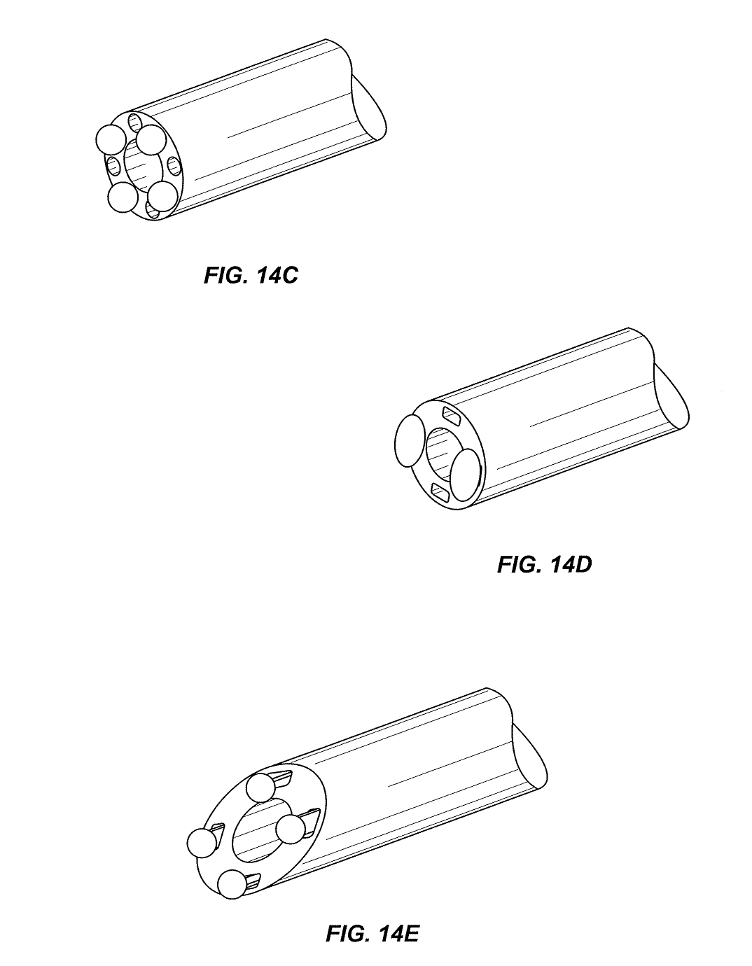

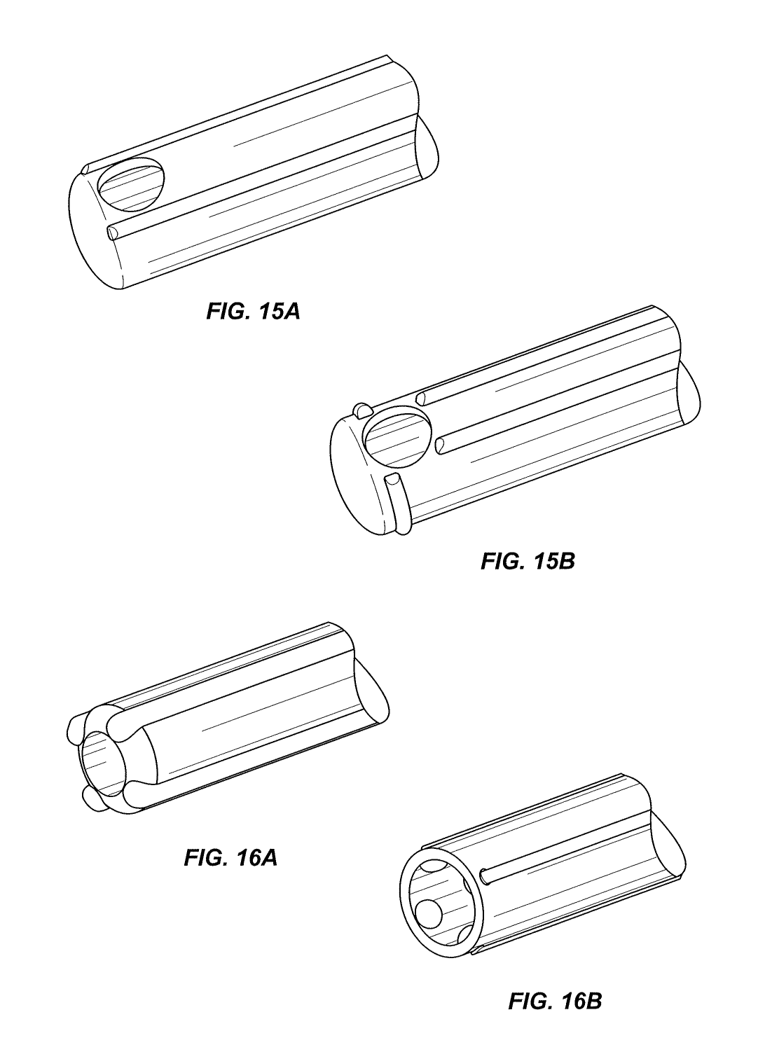

The engagement interface at the distal tip of the probe shaft may also be of various configurations, including various axial, lateral, and angled configurations for the electrodes and aspiration and/or irrigation lumens. Several examples are illustrated in FIGS. 14A-14E, 15A-15B, and 16A-16B. The probe shaft may further feature a soft atraumatic tip for retinal protection. Several of these configurations are pictured in FIGS. 13 and 15, for example. FIG. 14 illustrates additional embodiments, including embodiments having either two or four electrodes, and having a central aspiration lumen, radial lumens for irrigation or instrumentation, triangular pointed electrode tips directed radially toward the center of the sheathed orifice, spherical (or ellipsoidal) electrode tips circling the aspiration orifice. FIG. 14E illustrates a configuration in which the distal end of the probe shaft is beveled so as to form an oblique angle transverse to the shaft; the electrodes and aspiration orifice extend through the beveled end of the probe shaft. FIGS. 15A-15B illustrate shaft configurations featuring electrodes that are penned and/or plated onto a shaft exterior, as well as an aspiration orifice disposed on a side wall of the shaft, rather than through the distal tip of the shaft. FIG. 15-A illustrates two electrodes extending on either side of a side-wall orifice, while FIG. 15-B illustrates a configuration with four electrode “fingers” disposed around a side-wall orifice. In some configurations, such as those illustrated in FIGS. 16A and 16B, the tips of the electrodes may be formed so that the tips are closer to one another than the portions of the electrodes extending up the shaft, thus intensifying the electric field produced at the distal end of the probe. Of course, those skilled in the art will appreciate that the pictured embodiments are only exemplary; various other configurations will be described and discussed herein.

In some embodiments of the probe described above, the probe shaft has a diameter of less than 0.04 inches, and a length greater than one inch. In addition, it may be desirable for the probe shaft to have a bend resistance comparable to the stainless steel used in many conventional mechanical vitrectomy devices. However, included in this narrow shaft are features for passage of electrical conductors as well as one or more channels for the movement of fluid and effluent. Furthermore, the electrical conductors must be insulated from each other. Accordingly, fashioning a shaft of this complexity with the desired rigidity and reliability presents several engineering challenges.

The shaft may be fabricated from a dielectric material of high resistance, in some embodiments, to provide the required strength and insulating properties. Candidate materials include ceramics (or ceramic hybrids), quartz composites, pyrolytic carbon, sufficiently stiff thermoplastics, or composites of two or more of these. However, probe fabrication using such dielelectric materals with the necessary dimensions and with the features discussed above is a challenge. One approach to address these challenges is to create the shaft from an assemblage of small sections stacked, fitted or otherwise axially aligned with each other. Each small section may be formed from micromolding, by deposition processes, lithography, two photon polymerization or other suitable means, thereby maintaining stringent dimensional features along the entire length of the shaft. Modular sections may be held together by bonding or physical entrapment. This modular approach to fabricating the probe shaft facilitates the meeting of strict mechanical requirements as well as permitting tremendous flexibility in the probe shaft configuration.

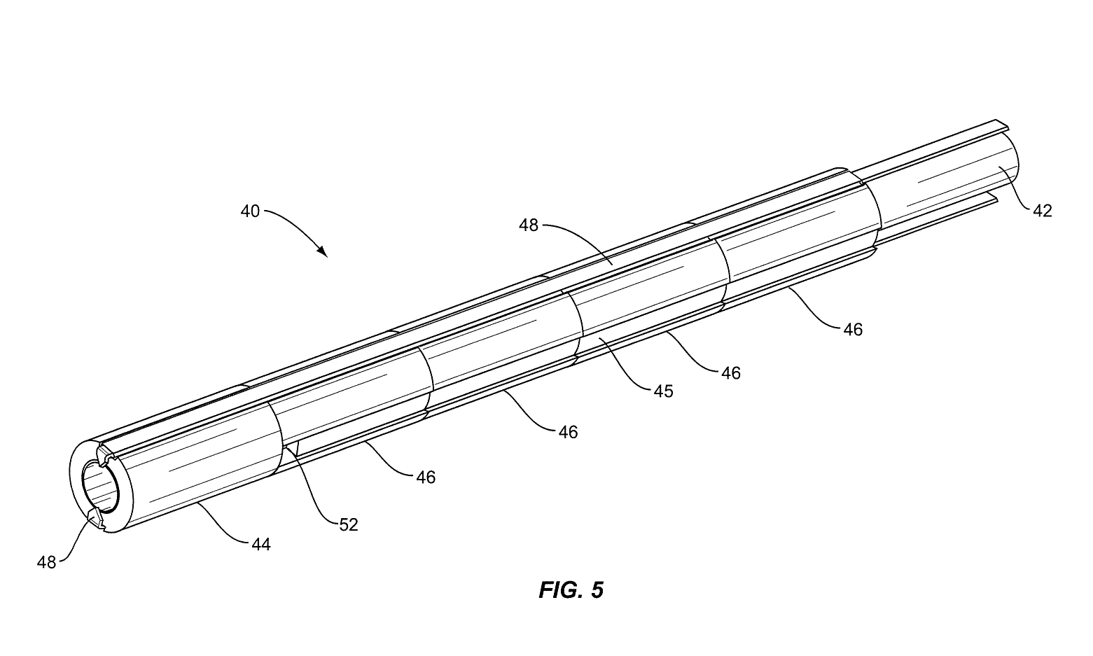

FIG. 4 illustrates several modular components that may be used to assemble an exemplary probe shaft. Modular shaft components, each of which may be formed from one or more of the materials discussed above, are fitted onto aspiration tube 42, which provides a central aspiration lumen from the distal end of the probe shaft to an aspiration line exiting the probe body. In particular, a modular end segment 44 and one or more additional modular shaft segments 46 may be fitted onto the aspiration tube 42. One or more of the modular segments may be bonded to the aspiration tube 42, in some embodiments, and/or to one another. One or more electrode units 48 may be fitted into longitudinal channels 45 running along the modular shaft components 44 and 46. In the pictured embodiment, the electrode unit 48 comprises a flat wire electrode, with a portion of the electrode surrounded by an insulating material 49. In the pictured embodiment, these longitudinal channels 45 are on the outside surfaces of the shaft components; in other embodiments, the longitudinal channels 45 may instead comprise tubular channels embedded within the shaft components.

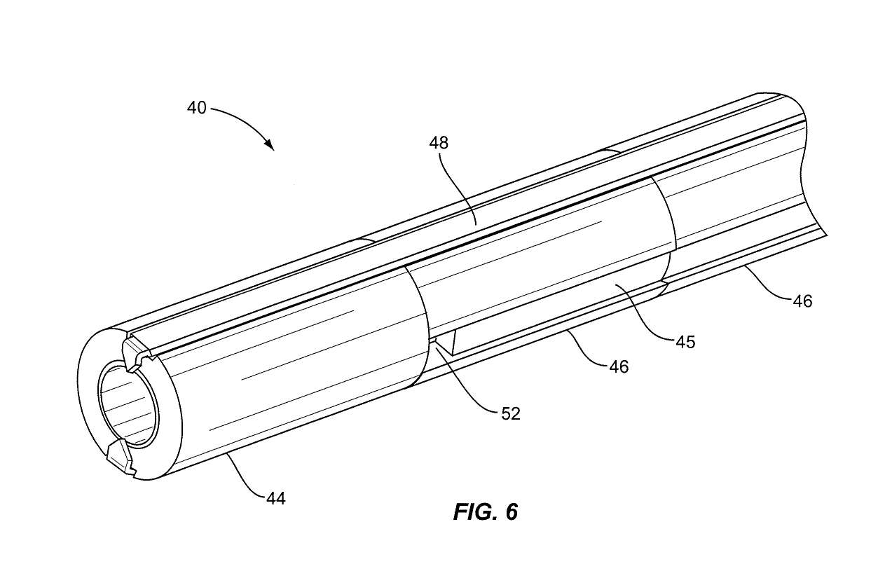

FIG. 5 illustrates a partially assembled probe shaft 40, built from the components pictured in FIG. 4. Probe shaft 40 thus comprises the aspiration tube 42, which fits inside a central longitudinal channel formed by the inner openings in end segment 44 and additional shaft segments 46. As pictured, two electrode units 48 are installed into the longitudinal channels 45 on the surface of the shaft segments; only the tip of one of the electrode units 48 is visible at the distal end of the probe shaft.

Each of the modular segments may have one or several longitudinal channels formed on its surface or within its body. These longitudinal channels may be adapted to accommodate an electrode unit, in some embodiments. In the assembly pictured in FIG. 5, the modular end segment 44 has two such longitudinal channels, each occupied by an electrode unit 48. Each of the additional shaft segments 46 has four longitudinal channels 45, two of which are aligned with the channels on end segment 44 and are occupied by an electrode unit 48. Those skilled in the art will appreciate that this ability to mix and match segments provides a great deal of flexibility in configuring a probe shaft, as the longitudinal channels may be used to contain electrodes or to form fluidic channels for irrigation or aspiration. Other uses for these channels are possible as well. For instance, one or more such channels may be used to hold an optical fiber (or other optical waveguide) extending from the probe body through the probe shaft to at or near the distal end of the shaft. This optical fiber may be used to provide a light source to the surgical site, in some embodiments, or to allow viewing of the surgical site in others, via a camera or optical instrument.

Still other features may be formed in or on one or more of the shaft segments. For instance, referring once more to FIG. 5, the longitudinal channel 45 that is not occupied by an electrode unit 48 may be used as an irrigation channel. Furthermore, because occasional engorgement of the aspiration channel when engaging vitreous tissue is possible, a shunt is provided between the irrigation conduits and the aspiration channel to dilute effluent vitreous fragments and allow for easier aspiration. Thus, in the embodiment pictured in FIG. 5, a shunting irrigation channel 52 is provided, allowing irrigation fluid to be shunted into the aspiration channel to improve the aspiration flow.

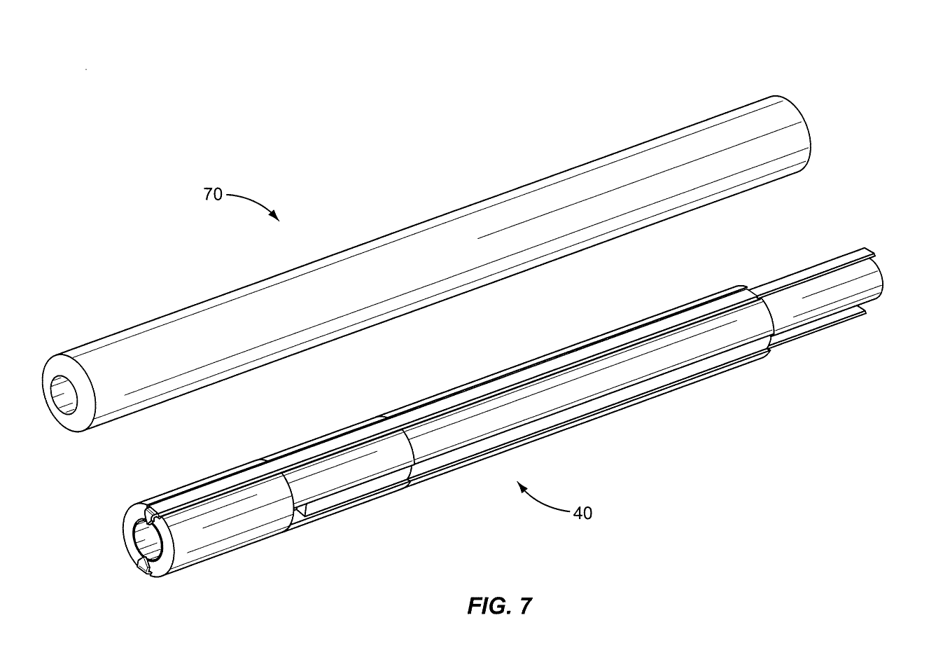

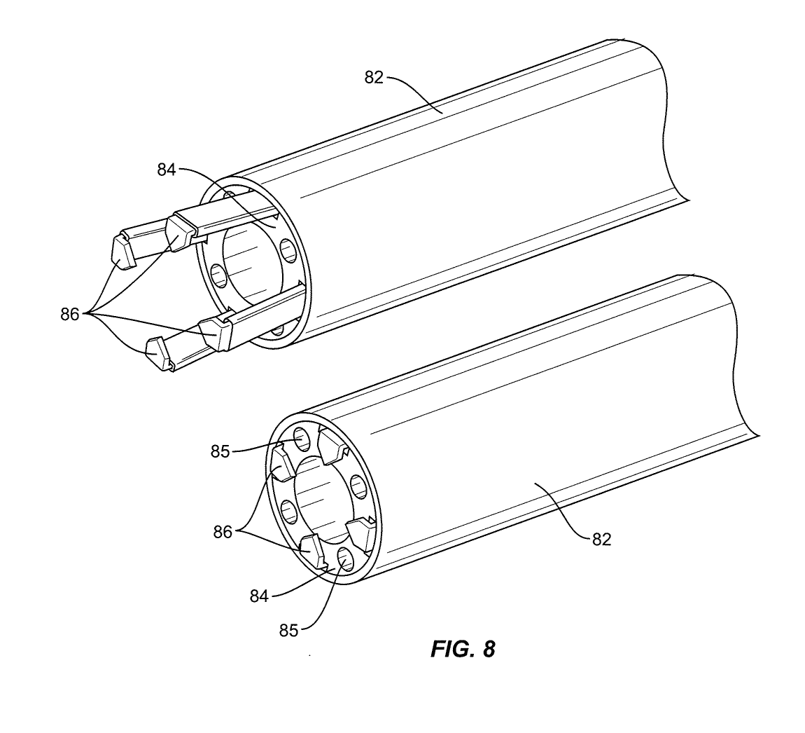

Further details of the probe shaft assembly 40 may be seen in FIG. 6, which provides a close-up view of the distal end of the probe shaft 40 as well as details of the shunting irrigation channel 52. In FIG. 7, a sheath 70 is pictured; the sheath 70 may be formed from a flexible material and is generally configured to slide over the assembled modular segments. When fully assembled, the end of the sheath 70 presents an atraumatic axial orifice, at the distal end of the probe shaft, to the tissue at the surgical site. Thus, with the application of the HIPEF energy to the surgical site through electrodes 48, near traction-free removal of vitreous and other ocular tissues from the posterior region of the eye may be achieved, without damaging the ultra-fine structure and function of the adjacent or adherent retina. Those skilled in the art will appreciate that with no moving parts, multiple probe tip embodiments are possible for a multitude of engagement situations.

In the Kovalcheck application, probes having two or more electrodes were discussed, but the distance between the distal tips of the electrodes in the probe were fixed. Those skilled in the art will appreciate that electric field strength is proportionally to the electrical potential between the electrodes and inversely related to the distance between electrodes. Thus, the shorter the distance, the stronger the field, given the same electric potentials. With a fixed electrode distance, changes in field strength are accomplished by changing the electrode potential (delivered pulse voltage).

In some embodiments of the probe shafts discussed herein, field strength may be changed by changing the distance between electrode tips by advancing or retracting the electrode wires within the probe shaft. This feature allows versatile manipulation of the applicator and the subsequent electric field strength, and may be combined with any of the other techniques or features disclosed herein, including, for example, the use of fluidic channels integral to the probe shaft to draw in and remove the affected volume, and/or to provide saline irrigation.

In some embodiments, the electrical conductor elements (electrodes) of the probe consist of flat, ribbon-like, spring-tempered wires that are slightly bent prior to assembly into the probe shaft. When the electrodes are assembled into the probe shaft, the bent wires are straightened but retain the memory of the bend. Under user control, the electrodes are movable axially within channels or lumens provided in the probe shaft, so that as the electrodes are advanced distally, the memory of the bend will cause the tips of the electrodes to move apart in a radial direction, thereby increasing the distance between the ends or tips of the electrodes. The extent of separation is related to the extent of axial movement out of the distal end of the probe shaft.

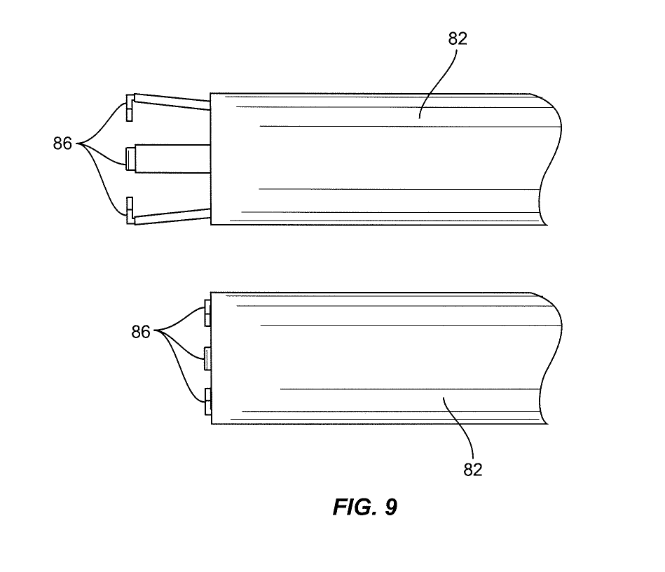

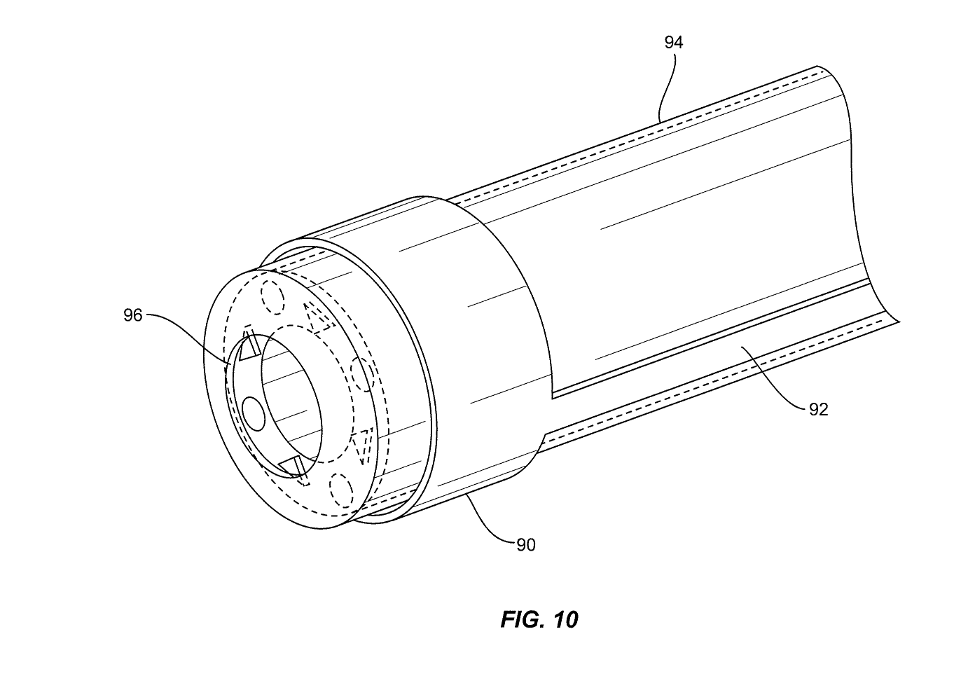

This is illustrated in FIGS. 8 and 9, which illustrate a probe shaft body 84, encased in a sheath 82. The probe shaft body has a central aspiration lumen, as well as one or other longitudinal lumens 85, each of which may be used for irrigation, or instrumentation. Electrodes 86 are configured to move longitudinally within channels on the probe body 84. When in an extended position, the tips of electrodes 86 move apart radially, as the electrodes “remember” their shape. As the electrode tips move apart, the strength of the pulse electric field generated between the electrodes decreases. In contrast, as the electrodes are retracted into the probe shaft, the distance between electrode tips decreases. If an electric potential is applied to the electrodes during retraction, then the field strength between electrode tips would subsequently increase as the wires were retracted.

Thus, the field strength can be actively increased or decreased by simply moving the electrodes axially to and from within the lumens of probe. The movement of the electrodes may be under the manual control of the operator, e.g., using a trigger or slider mechanism, or may be motorized, with control via electrical control circuitry. Those skilled in the art will appreciate that the extensible electrode tips, while primarily designed for delivery and development of a field between the tips of the exposed electrodes, may also act, during axial retraction, as a grasper or retriever for engaging and moving the tissue exposed the field between the electrode tips. While this movable electrode feature is described with the electrode tips moving apart during axial advancement, the bend in the electrode wires may be reversed such that the electrode tips would move closer together during axial advancement creating stronger fields the more distal from the aspiration orifice. While primarily designed for delivery and development of a field between the tips of the exposed electrodes, the exposed tips may also act, during axial advancement, as a grasper or retriever for engaging and moving the tissue exposed the field between the electrode tips.

As discussed in the Kovalcheck application, the energy delivered to the tissue via high intensity ultrashort electrical pulses is insufficient to create electron avalanche and thus is considered inherently safe. Nevertheless, additional grounding features may be added to the probe shaft designs discussed herein, to further constrain the electrical fields produced by the probe. These additional features inhibit or reduce the possibility of electrical current escaping or straying into tissues that do not require treatment.

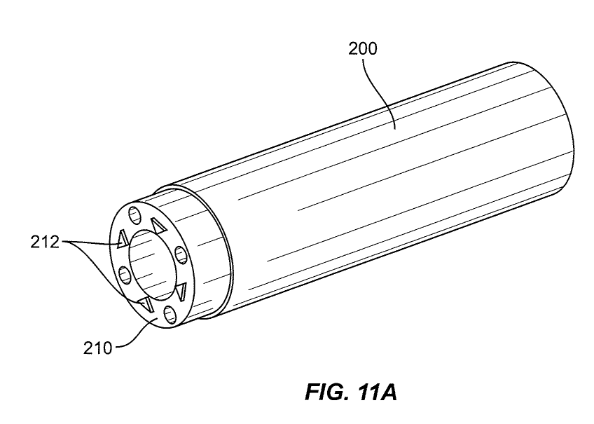

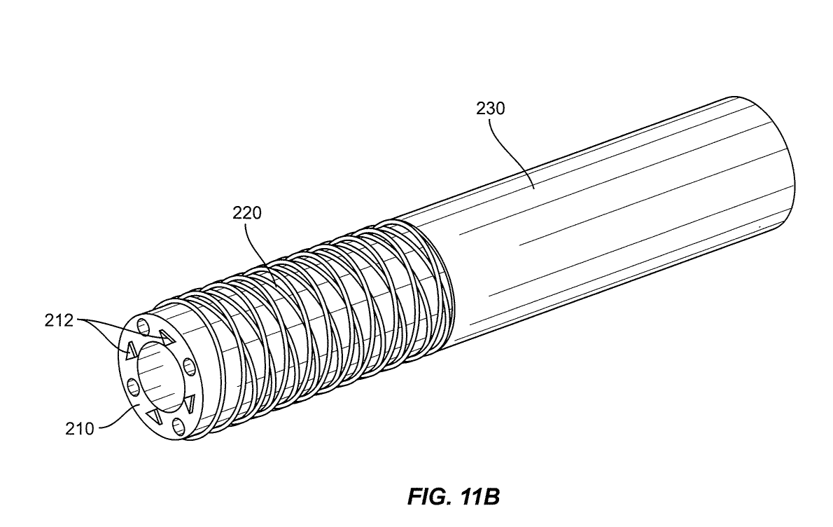

Several embodiments are possible. In one embodiment, pictured in FIG. 10, a conductive metallic ring 90 is placed at or near the distal tip 96 of the probe shaft 94. An additional electrical conducting wire 92 is incorporated into or onto the probe shaft; the conducting wire 92 connects the ring 90 to system ground, e.g., via a cable attached to the probe body. In a second embodiment, pictured in FIG. 11A, the probe shaft has a conductive outer surface layer 200, which terminates near the end of the probe shaft and communicates proximally with system ground. The dielectric multi-lumen body 210 of the probe shaft (which may be fabricated according to any of the techniques discussed above) serves to insulate the conductive outer surface layer 200, which may comprise a metal tube, sheath, or plating, from the conductive electrode tips 212.

In a similar embodiment, pictured in FIG. 11B, a metallic wire braid 220 is incorporated into or onto the dielectric polymer of the probe shaft 230. The braid 220 terminates near the distal end of the probe shaft, and communicates with system ground on the proximal end of the probe shaft. As with the other embodiments, the grounding means is insulated from the high intensity pulse delivery electrodes.

Another design consideration for the various HIPEF probe devices discussed above is the shape of the electrodes. These electrodes, which may have diameters of less than one millimeter in some embodiments, may be configured with shapes designed to achieve maximum field strength along with a homogeneous field, without localized field enhancement. The particular shape of the electrodes has a direct effect on the features of the generated electric field; the features of the generated electric field may in turn have a significant effect on the practical performance of the probe. FIGS. 14, 15 and 16 illustrate several electrode shape embodiments with two and four electrodes.

In general, the performance of a HIPEF probe is linked to the electric field it produces at its tip. The produced electric field is dependent on the form and number of electrodes that are used to produce it. Each configuration (electrode form and number) produces a different electric field with different properties. Comparing these electric fields to find an ‘optimal’ shape can be difficult, because no ranking exists that weights one property over another. One approach is to simply experiment with various configurations to find an acceptable configuration. Another approach, as detailed more fully below, is to determine a figure of merit for an electrode configuration, so that one configuration can be compared to another.

In the discussion that follows, the term “shape” is used to refer to a specific arrangement of two or more electrodes and their specific physical form. “E_{ideal}” is used to refer to an ideal electric field strength across the aspiration lumen 122, assuming a uniform field. This is the maximum electric field strength that is theoretically achievable. “E_{avg}”, on the other hand, refers to the average electric field strength averaged over the aspiration lumen, while “Eₚₑₐₖ” refers to the peak electric field strength anywhere in the considered area and “σ_{Eavg}” is the standard deviation of the electric field from the average, measured over the aspiration lumen. “R” is used in the discussion that follows to refer to the maximum extent (transverse to the tip of the probe) of the electrodes, while “r” refers to the electrodes' minimum extent. Finally, “α” is the figure of merit for a given shape, determined according to the techniques discussed below.

The figure of merit α is an attempt to objectively judge the performance of a shape. It combines various features of the electric field generated by a given shape, and assigns a number to it. The higher the figure of merit for a particular shape, the better its performance. Several features of the electric field of a shape are of interest, and may be reflected in the figure of merit. First, the average electric field (E_{avg}) in the region where it matters (i.e., over the aspiration lumen) is reflected in the figure of merit. As the electric field does all the work, it should be as high as possible for a given pulse amplitude. In other words, a higher average electric field is better. Ideally, the average electric field strength is close to the ideal (maximum, highest achievable uniform) electric field strength (E_{ideal}).

The variation of the electric field (σ_{Eavg}) in the same region of interest, expressed as standard deviation, may also be reflected in the figure of merit. Large variations indicate regions of high and low electric field strength. Regions of low electric field strength result in less treatment in that low field region, whereas regions of high fields may lead to bubble formation. Accordingly, a lower standard deviation is better. Ideally, the standard deviation is zero. The peak electric field strength anywhere in the considered area may also be considered. As already noted, high electric fields may lead to bubble formation, and may also lead to dielectric breakdown. Thus, lower peak electric field strength (for a given average field strength) is better. Ideally, the peak electric field strength is equal to the average electric field strength.

The above mentioned values may be combined in the following equation to yield a figure-of-merit:

$$α = \frac{{E}_{avg}}{{E}_{ideal}} \cdot \frac{{E}_{avg}}{{E}_{peak}} \cdot ( 1 - \frac{{σ}_{Eavg}}{{E}_{avg}} ) .$$

The left-most term of the right-hand-side,

$$\frac{{E}_{avg}}{{E}_{ideal}} ,$$

expresses how much of the ideal electric field strength is achieved. This ratio can be larger than unity if there are large peak electric fields in the region of interest. The second term of the right-hand-side,

$$\frac{{E}_{avg}}{{E}_{peak}} ,$$

expresses how much field enhancement over the average is produced by the shape. This ratio can never be larger than unity, and is included as the inverse of the actual field enhancement

$$( i . e . , \frac{{E}_{peak}}{{E}_{avg}} ) ,$$

to effectively penalize large localized enhancements of the electric field over the average intensity. The last term,

$$\frac{{σ}_{Eavg}}{{E}_{avg}} ,$$

relates the standard deviation of the electric field to its average (for the region of interest, not the entire region). This term accounts for the achieved uniformity of the electric field. The higher this ratio is, the worse the uniformity. By subtracting this ratio from 1, a unity value is obtained for a uniform field. This ratio can never be larger than unity.

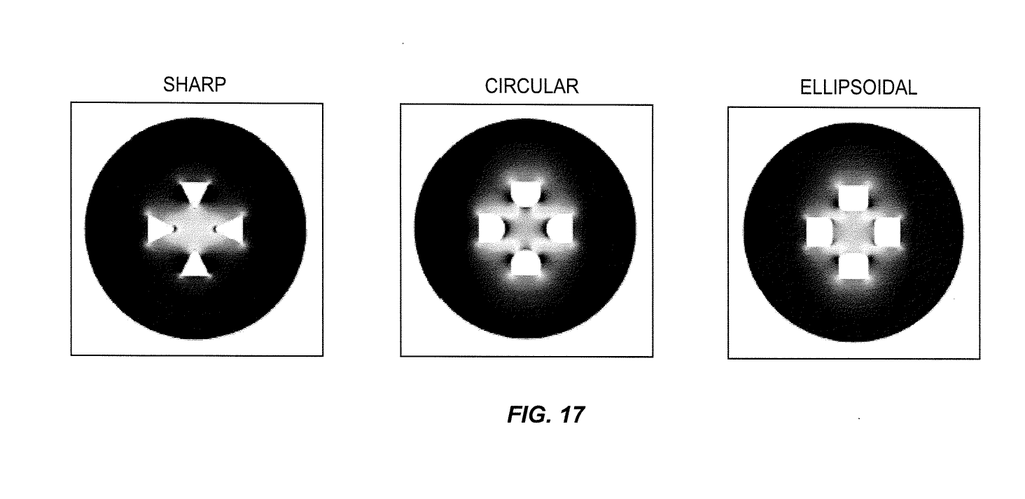

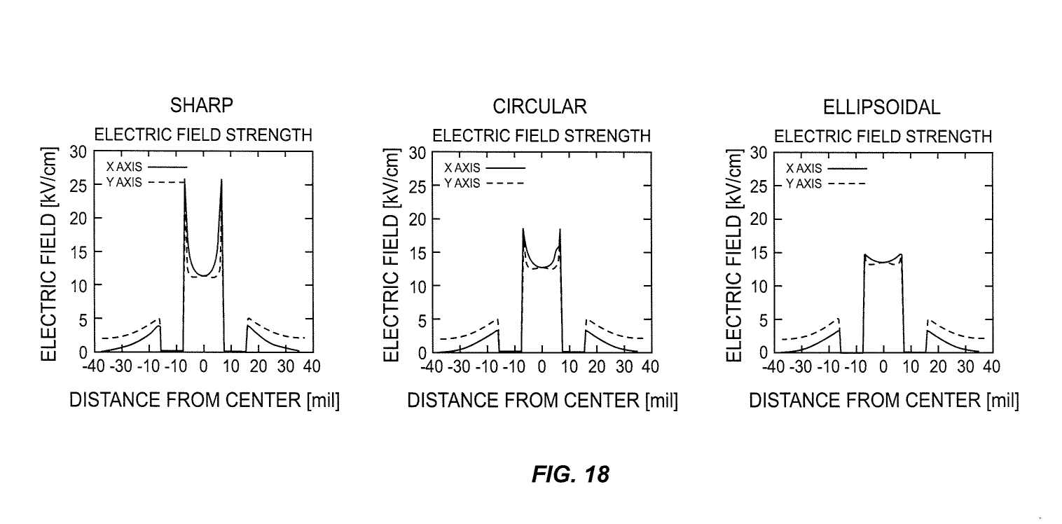

An ideal shape would result in a figure of merit with a value of 1. In other words, the ideal shape would produce an electric field with the maximum possible electric field (for a given pulse amplitude) with no variations in the field strength in the region of interest, and no field enhancement anywhere in the considered area. Of course, practical configurations will generally have a some non-uniformities. Exemplary electric field intensities for several electrode configurations, having electrode tips with triangular, circular, and ellipsoidal cross-sections, respectively, are illustrated in FIG. 17 and FIG. 18. Those skilled in the art will appreciate that the triangular electrode tips yield the highest maximum field strength, given the four-electrode configuration pictured, but also exhibit the highest variation in electric field over the area of interest. The other configurations yield a lower maximum field strength, but with better uniformity across the area of interest. The figure of merit discussed herein provides a useful means for comparing the relative fitness for purpose of various configurations.

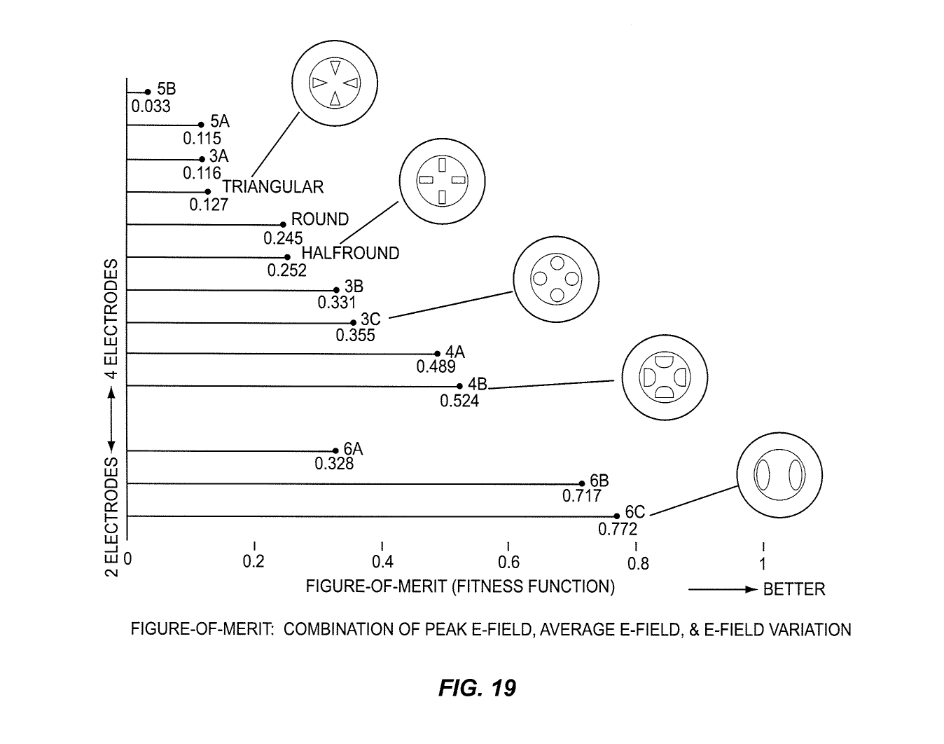

As shown in FIG. 19, figures of merit have been determined for several practical electrode shapes. In particular, a figure of merit of 0.772 for an exemplary two-electrode configuration has been determined, while a figure of merit of 0.355 has been determined for a four-electrode configuration. Evaluation of various shapes reveals that the ideal form of the electrodes varies depending on the ratio of the maximum shape dimension (R) to the dimension of the aspiration lumen inside the electrodes (r). Of course, the exemplary figures of merit given above are only valid for a particular ratio. Nevertheless, the figure of merit discussed above may be used to compare dramatically dissimilar electrode configurations to one another.

With the several exemplary embodiments discussed above and pictured in FIGS. 1 to 19 in mind, those skilled in the art will appreciate that the preceding descriptions of various methods and apparatus for controlling the application of high-intensity pulsed electric field energy during eye surgery were given for purposes of illustration and example. Thus, aspects of the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A probe for applying high-intensity pulsed electric fields to intraocular tissue, comprising a probe shaft assembled from a modular end segment and one or more additional modular shaft segments, each of the modular end segment and one or more modular shaft segments comprising at least two longitudinal channels adapted to accommodate an electrode unit.

2. The probe of claim 1, wherein each of the modular end segment and one or more modular shaft segments comprises a central longitudinal channel, and wherein the probe shaft further comprises an aspiration tube disposed within the central longitudinal channel.

3. The probe of claim 1, wherein at least one of the modular end segment or the modular shaft segments is formed from a ceramic or ceramic hybrid material.

4. The probe of claim 1, wherein at least one of the modular shaft segments comprises a shunting irrigation channel.

5. The probe of claim 1, wherein the modular end segment and the one or more modular shaft segments each comprise at least one longitudinal channel configured to serve as an irrigation channel.

6. The probe of claim 1, wherein the modular end segment and the one or more modular shaft segments each comprise at least one longitudinal channel configured to accommodate an optical waveguide.

7. The probe of claim 1, wherein the probe shaft further comprises a sheath fitted over the modular end segment and the one or more modular shaft segments.

8. The probe of claim 7, wherein the sheath comprises a conductive element substantially encircling the probe shaft at or near the distal end of the probe shaft, wherein the conductive element is adapted for connection to electrical ground through a probe body.

9. The probe of claim 8, wherein the conductive element comprises braided wire.

10. The probe of claim 1, wherein at least one of the modular end segment or the modular shaft segments comprises a by-pass orifice adapted to permit fluid flow between first and second longitudinal channels in the segment.

11. The probe of claim 1, wherein the electrode units in the at least two longitudinal channels are configured to slide along the longitudinal channels, under user control, from a first configuration, in which a tip of each electrode unit is within or proximal to a distal end of the probe shaft, to a second configuration, in which the tip of each electrode unit is extended from the distal end of the probe.

12. The probe of claim 11, wherein the electrode units are pre-bent and arranged within the corresponding longitudinal channels such that the tips of the electrode units move apart in a radial direction as the electrode units are moved from the first configuration to the second configuration.

13. The probe of claim 1, further comprising an amplifier circuit configured to amplify an electrical pulse supplied to the probe prior to application to the eye via at least one of the electrode units.

14. The probe of claim 1, further comprising an integral termination circuit configured to receive electrical pulses supplied to the probe via a transmission line, wherein the termination circuit is substantially matched to the characteristic impedance of the transmission line.

15. The probe of claim 1, further comprising a pulse-shaping circuit configured to shorten the rise time of one or more electrical pulses supplied to the probe prior to application of the shaped pulses to the eye.

16. An apparatus for applying high-intensity pulsed electric fields to intraocular tissue, the apparatus comprising first and second probes configured to be applied to an eye, the first probe comprising at least one electrode adapted for delivery of high-intensity pulsed electric field energy to intraocular tissue, at least one of the probes comprising an optical waveguide extending at least substantially to the distal end of the at least one of the probes, and the second probe comprising at least one intraocular surgical feature selected from the group consisting of:
an optical waveguide extending at least substantially to the distal end of the second probe;
an aspiration lumen;
an irrigation lumen; and
one or more additional electrodes.

17. The apparatus of claim 16, wherein the first probe comprises a probe shaft assembled from a modular end segment and one or more additional modular shaft segments, each of the modular end segment and one or more modular shaft segments comprising at least two longitudinal channels adapted to accommodate an electrode unit.

18. The apparatus of claim 16, further comprising a third probe configured to be applied to the eye, the third probe comprising at least one intraocular surgical feature selected from the group consisting of:
an optical waveguide extending at least substantially to the distal end of the second probe;
an aspiration lumen; and
an irrigation lumen.

19. The apparatus of claim 16, wherein the first probe comprises a probe shaft having a distal end configured for insertion into the eye and further comprises a conductive element substantially encircling the distal end of the probe shaft, wherein the conductive element is adapted for connection to electrical ground through a body of the first probe.

20. The apparatus of claim 16, wherein the first probe comprises two or more electrodes configured to slide along longitudinal channels in a shaft of the first probe, under user control, from a first configuration, in which a tip of each electrode unit is within or proximal to a distal end of the probe shaft, to a second configuration, in which the tip of each electrode unit is extended from the distal end of the probe.

21. The apparatus of claim 20, wherein the electrode units are pre-bent and arranged within the corresponding longitudinal channels such that the tips of the electrode units move apart or closer in a radial direction as the electrode units are moved from the first configuration to the second configuration.

22. The apparatus of claim 16, wherein the first probe comprises an amplifier circuit configured to amplify an electrical pulse supplied to the probe for application to the eye via the at least one electrode.

23. The apparatus of claim 16, wherein the first probe comprises an integral termination circuit configured to receive electrical pulses supplied to the probe via a transmission line, wherein the termination circuit is substantially matched to the characteristic impedance of the transmission line.

24. The apparatus of claim 16, wherein the first probe comprises a pulse-shaping circuit configured to shorten the rise time of one or more electrical pulses supplied to the probe prior to application of the shaped pulses to the eye.

25. A probe for applying high-intensity pulsed electric fields to intraocular tissue, the probe comprising:
a probe shaft having at least one longitudinal aspiration lumen disposed therein and having a distal end configured for insertion in an eye, the probe shaft further comprising an aspiration orifice disposed at or near the distal end and in communication with the aspiration lumen; and
two or more electrodes disposed in or on the probe shaft and terminating at or near the end of the probe shaft;
wherein each of the terminal ends of the two or more electrodes is configured to have a cross-selection selected from the group consisting of:
a substantially circular cross-section;
a substantially triangular cross-section;
a substantially rectangular cross-section; and
a substantially ellipsoidal cross-section.

26. The probe of claim 25, wherein each of the terminal ends of the two or more electrodes is configured to have a substantially circular cross-section.

27. The probe of claim 25, wherein each of the terminal ends of the two or more electrodes is configured to have a substantially triangular cross-section.

28. The probe of claim 25, wherein each of the terminal ends of the two or more electrodes is configured to have a substantially rectangular cross-section.

29. The probe of claim 25, wherein each of the terminal ends of the two or more electrodes is configured to have a substantially ellipsoidal cross-section.

30. The probe of claim 25, wherein the aspiration orifice is disposed on a sidewall of the probe shaft.

31. The probe of claim 25, wherein the distal end of the probe is beveled at an oblique angle, and wherein the aspiration orifice is disposed on the beveled distal end.
